(19) United States
(12) Patent Application Publication
(10) Pub. No.: US 2016/0251684 A1
(43) Pub. Date: Sep. 1, 2016

(54) UTILIZATION OF INTESTINAL BACTERIA FROM SLAUGHTERED ANIMALS IN INDUSTRIAL PROCESSES AND WASTE TREATMENT

(71) Applicant: Eino Elias HAKALEHTO, Kuopio (FI)

(72) Inventor: Eino Elias Hakalehto, Kuopio (FI)

(21) Appl. No.: 15/027,989

(22) PCT Filed: Oct. 8, 2014
(86) PCT No.: PCT/FI2014/000026

§ 371 Date: Apr. 7, 2016

(30) Foreign Application Priority Data
Oct. 8, 2013 (FI) 20130286

(51) Int. Cl. C12P 7/52; C12P 7/18; C12P 7/04; C12M 1/00

(52) U.S. Cl. CPC C12P 7/52; C12M 29/00; C12P 7/18; C12P 7/04

(57) ABSTRACT

Slaughter waste normally contains considerable amounts of so-called stomach manure. Its biotechnical treatment rapidly decreases the waste problem. With the help of the method and device according to this invention stomach manure is first used as a biocatalyst in a process that produces mannitol. After that the lactic acid that at the same time farms into the slaughter waste is converted with the help of microbes into propionic acid or other useful compounds.

### BACKGROUND

Mannitol that forms as a result of bacterial metabolism normally comes from fructose. In nature, fructose is found, e.g. in hemicellulose (for example in wood material), and in various parts of plants, such as in fruits. Wastes and byproducts that contain fructose are thus well suitable as raw materials for mannitol production.

Fructose can also be formed from raw materials that contain glucose (cellulose, starch, etc.). For example, the starch from corn, barley, potato, or some other plant, can be split, with the help of amylase and amyloglucosidase entzymes, into glucose, which is then, with the help of glucose-isomerase enzymes, into fructose. This is the process used in the production of High Fructose Corn Syrup. It has been developed into an industrially viable product and High Fructose Corn Syrup is an important sweetener. Correspondingly, different sugar industry fractions can be enzymatically processed and their fructose content and respectively their sweetening value can thus be increased.

The broad use of fructose in foodstuffs has lately been hindered by the uncertainty of its healthiness as a component and a sweetener in human nutrition. Fructose breaks down in the body only in the liver and its excessive use has been shown to cause obesity.

Stomach manure is the content of the stomach that forms into the rumen in bovine and other ruminants. In slaughter houses the amount of stomach manure is approximately 50 kg per one bovine. Thus in a slaughterhouse that slaughters 200 animals a day, about 10 tons of stomach manure is gathered per day. Stomach manure is formed naturally also from the slaughtering of other than bovine animals. The utilization of this waste for example by composting or by producing biogas, requires considerable space and thus expensive investments. Thus the economic benefit and increase of cleaning power gained with a fast biotechnical process is meaningful.

Stomach manure microbes exist also in the manure of other slaughter animals. Stomach manure or manure can be combined with other wastes in different waste treatment processes.

### DESCRIPTION

When mannitol is produced with microbes, lactic add bacteria are normally the production organisms that are used. They are known safe production organisms, but it can be challenging to find a suitable strain for an efficient enough conversion of fructose to mannitol.

Using a method according to this invention, fructose or waste rich in fructose, a byproduct or a supplement is added into slaughter waste. This material contains for example at a bovine slaughterhouse a large amount of so-called stomach manure which, in practice, is the content of the rumen and other stomach content of the slaughtered animals. About 50 kg of this is formed per animal.

With the help of stomach manure microbes or other microbes added to them, also other different waste materials that are integrated into the process, can be utilized.

With the help of their common metabolism the microflora of the rumen that is contained in the stomach manure is capable of changing into mannitol the fructose that is in the waste or byproduct or added into it. At the same time the lactic acid bacteria form lactate which lowers the pH-value of the process liquid. With the help of certain lactic acid bacteria and other bacteria, such as Lactobacillus xylosus, Propionibacterium shermani and Propionibacterium acidipropionici lactate can be converted into propionate, i.e. propionic acid. The Clostridium propionicum bacteria form propionic acid, acetate, ammonia and carbon dioxide from L-alanine. The optimal pH for the formation of propionic acid is often 6,5. In addition to these products mentioned above, also several other organic and inorganic compounds are formed in the process. The ammonium salts formed in it can be precipitated apart from the process liquid and collected for use as a fertilizer together with the residue of the process liquid, or it can be separated from it. Thus with a method according to this invention fertilizers may be gained from stomach manure or other waste.

The mixed microbe population or mixed culture of the rumen can also convert glucose found in waste material or byproduct or other raw material, into mannitol. This requires that glucose is first enzymatically converted into fructose. in this case the same glucose-isomerase enzymes that are used in the production of fructose syrup (HFCS, high fructose corn syrup, GFS, glucose-fructose syrup, high-fructose maize-syrup, glucose/fructose) can be used.

In practice HFCS is produced from raw material that is rich in starch, mainly from corn. Also many other plants such as potato and cereal contain significant amounts of plant starch. The following enzymes participate in the reaction:
1. Alpha-amylase (forms short chain oligosaccharides from starch)
2. Glucoamylase (splits oligosaccharides into glucose)
3. Glucose-isomerase (converts about 42% of fructose and 50-52% of glucose and some other sugars into the mixture)

Using a method according to this invention fructose is produced from glucose if necessary with the help of industrial enzymes presented above or with the help of other industrial enzymes that release glucose. For example with the help of lactic acid bacteria mannitol can further be obtained from fructose. With the help of this process, the added value of many biomasses, side streams and waste materials can thus be increased. Glucose is a molecular structure that is commonly present in natural materials such as starch, cellulose and hemicellulose. Fructose is found e.g. in the wastes and side streams of sugar, fruit and berry industries. Naturally it is clear that in this case microbe cells or other cells, tissue, cell parts or similar may function as biocatalysts, in addition to or along with enzymes or as sections that replace them.

Because the potential harmfulness of extensive use of fructose has been raised in recent discussion related to health, nutrition and chronic illnesses, it is advantageous to biotechnically refine fructose further to another useful form. Contrary to for example glucose, fructose does not metabolize in all the cells of the body, only in the liver. If excessive amounts of fructose are used this involves e.g. obesity and abdominal obesity risks and also the risks of fatty liver and other liver diseases. The energy content of mannitol is fairly low compared to fructose and glucose and many other molecules but it has considerable value of use e.g. in lozenges, chewing gums, medicinal products, pastries etc. as an additive and sweetener that improves product quality, such as taste, freshness etc. Also in this respect it is justified to convert fructose, glucose and other sugars to mannitol.

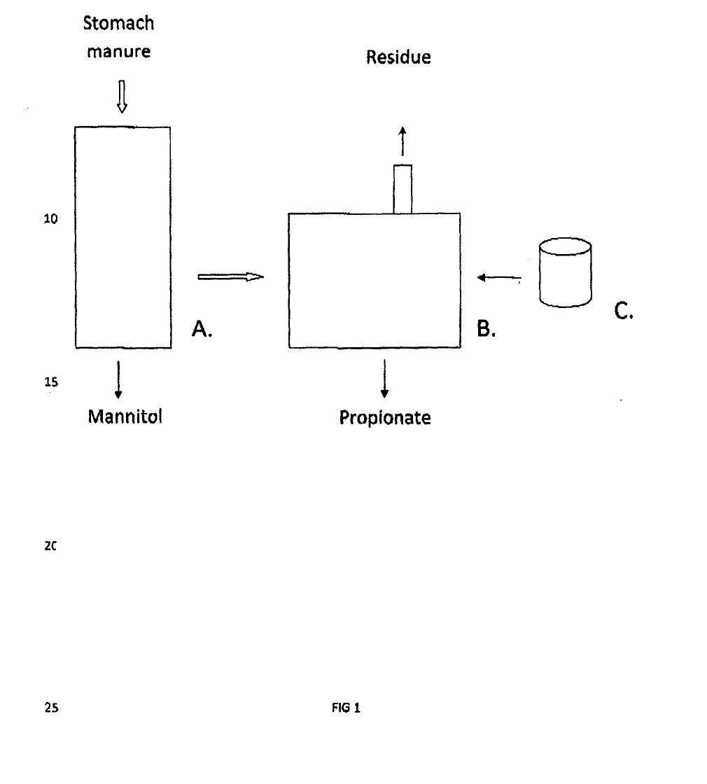

When sucrose (consists of glucose and fructose) was added into slaughter waste (stomach manure etc.), large amounts of mannitol formed into it. When the pH was not tried to be adjusted or kept at a certain pH level, contrary to what is normally advantageous in biotechnical processes, the yield was close to the theoretical maximum level being approximately 18 mg/ml. When the pH level was adjusted to be between 5 and 6,5, the corresponding production level was only 3 mg/ml. The high level of mannitol that occurred when the pH level was not adjusted during the biotechnical process was an unexpected reaction of the normal microflora of the rumen. A model for the implementation of the production process of mannitol is presented in FIG. 1.

If, as described above, the pH was not tried to be adjusted or stabilized, considerable amounts of lactate, i.e. lactic acid formed into the process liquid. An advantageous means of exploiting this is to convert it with the help of lactic acid bacteria into propionate which has a high energy content and which may be used as silage or feed for animals kept for fur or as food for other animals or even as an additive or preservative for human nutrition. It can also be used to prevent the deterioration of timber and structures and the icing of road surfaces.

An alternative way to process biomass based suspension or liquid is anaerobic acetone-butanol fermentation. The growth of clostridia can be accelerated by leading carbon dioxide into the gas solution or process liquid. Because clostridia can withstand up to 100% carbon dioxide content while growing a culture, this can be used to speed up the reaction. Clostridia can also be used to produce other organic acids such as butyric acid and valeric acid.

An installation to exploit the method according to this invention consists of A. a mannitol reactor, 8. a production pool for propionic acid formation and C. a seed fermenter (FIG. 1).

1-17 (canceled)

18. A method of utilizing intestinal bacteria, the method comprising the steps of:
preparing a process liquid by adding a sugar selected from the group consisting of monosaccharides, disaccharides, and their polymers into manure selected from the group consisting of stomach manure of a slaughtered animal, manure produced by an animal, and refinery sludge; and
increasing productivity or production levels of desired biotechnical products in the process liquid.

19. The method according to claim 18, wherein the sugar is sucrose, glucose, fructose, and a mixture of any of sucrose, glucose and fructose.

20. The method according to claim 19, wherein the sugar is glucose and fructose that is produced enzymatically as a result of decomposition of starch, cellulose, hemicellulose or other macromolecule.

21. The method according to claim 20, wherein that an amount of the fructose is added by enzymatic means.

22. The method according to claim 21 further comprises the step of converting the sugar in the manure into mannitol using lactic acid bacteria in the manure.

23. The method according to claim 22 further comprises the step of lowering a pH value of the process liquid by changing the glucose into lactic acid by at least the lactic acid bacteria and microbes performing acetic acid fermentation, which simultaneously prevents decomposition or metabolization of the mannitol that has formed into the process liquid.

24. The method according to claim 23, wherein the pH value starts to be raised by adding a base when at least most of the fructose has converted into mannitol.

25. The method according to claim 24 further comprises the step of removing the mannitol by crystallization prior to raising the pH value.

26. The method according to claim 25 further comprises the step of heating the process liquid by leading a gas emission containing one of CO and CO₂ into the process liquid, wherein the gas emission is formed from a combustion reaction.

27. The method according claim 26 further comprises the step of converting the lactic acid formed by the acetic acid fermentation to propionate.

28. The method according to claim 27, wherein the propionate is propionic acid, and wherein Clostridium bacteria is used in converting the propionic acid into an organic acid or an alcohol.

29. The method according to claim 27 further comprises the step of adding at least one bacterial strain into the manure for assisting in changing a part of the lactic acid into the propionate, wherein the part of the lactic acid is collected for burning or for chemical utilization as a preservative in secondary products.

30. The method according to claim 23, wherein the sugar is added to the manure after adjusting a humidity percentage of the manure in a reactor.

31. The method according to claim 30 further comprises the steps of: removing the mannitol from the process liquid;
leading a residue of the process liquid into a container; and
adjusting the pH value and temperature of the process liquid in the container for production of propionic acid.

32. The method according to claim 31 further comprises the step of adjusting microbial levels of the process liquid using microbial inoculums that is inoculated from a inoculation fermentor, wherein the inoculation fermentor has a dosing pump.

33. The method according to claim 1 further comprises the step of precipitating ammonium salts formed from the manure in the process liquid to fertilizers.

34. An apparatus for utilizing intestinal bacteria, said apparatus comprising:
a reactor having a configuration capable of containing a process liquid including manure and a sugar, and adjusting a humidity percentage of the manure in the reactor;
a container having a configuration capable of receiving a residue of the process liquid from the reactor, and adjusting a pH value and temperature of the residue suitable for production of propionic acid; and
an inoculation fermentor having a dosing pump, wherein the inoculation fermentor having a configuration capable of inoculating microbial inoculums therein, and wherein the dosing pump has a configuration capable of pumping the microbial inoculums to the reactor.
